(19) United States
(12) Patent Application Publication
(10) Pub. No.: US 2022/0175843 A1
(43) Pub. Date: Jun. 9, 2022

(54) Treatment of Microvascular Disorders with Mesenchymal Stem Cells and Their Exosomes

(71) Applicant: SYMBIOCELL TECH, LLC, Salt Lake City, UT (US)

(72) Inventors: Christof Westenfelder, Salt Lake City, UT (US); Anna Maddock Gooch, Salt Lake City, UT (US)

(21) Appl. No.: 17/602,072

(22) PCT Filed: Apr. 8, 2020
(86) PCT No.: PCT/US2020/027278

§ 371 Date: Oct. 7, 2021

Related U.S. Application Data
(60) Provisional application No. 62/830,807, filed on Apr. 8, 2019.

(51) Int. Cl. A61K 35/28

(52) U.S. Cl. CPC A61K 35/28

(57) ABSTRACT

The present disclosure relates to compositions for the treatment of microvascular diseases. Such compositions may comprise mesenchymal and/or adipose stem cells; and/or exosomes isolated from mesenchymal and/or adipose stem cells. Further included are methods of treating microvascular disease using the compositions and methods of making the compositions.

### CROSS-REFERENCE TO RELATED APPLICATIONS

This application is a national phase entry under 35 U.S.C. § 371 of International Patent Application PCT/US2020/027278, filed Apr. 8, 2020, designating the United States of America and published in English as International Patent Publication WO 2020/210363 on Oct. 15, 2020, which claims the benefit under Article 8 of the Patent Cooperation Treaty to U.S. Provisional Patent Application Ser. No. 62/830,807, filed Apr. 8, 2019, the entireties of which are hereby incorporated by reference.

### FIELD

This application relates generally to the treatment of a wide range of microvascular disorders with Mesenchymal Stem Cells and their Exosomes, administered in combination or individually. A large number of acute and chronic diseases are characterized, at both early and advanced stages, by the development of microvascular pathology that mediates progressive organ injury, organ loss, morbidity and mortality. Therapeutic targets for the here disclosed technology include both Type 1 and Type 2 Diabetes mellitus and their multiorgan complications (e.g. kidneys, eyes, heart, neurological and others), vasculitides, auto-immune diseases, sepsis, acute and chronic kidney diseases, cardiovascular and neurological diseases, solid organ transplantation, vascular rejection, wound healing, atherosclerosis, aging, and various degenerative retinal diseases. More specifically, disclosed embodiments relate to the therapeutic use of Mesenchymal Stem Cells from various sources and their Exosomes, in combination or individually, to repair and protect and stabilize the diseased microvasculature of a subject and thereby improve outcomes and survival.

### BACKGROUND

A large number of common, acute and chronic diseases are characterized, at both early and advanced stages, by the development of similar microvascular pathologies that lead to progressive organ injury, organ loss, morbidity, and mortality. The intrinsic capillary/microvascular network of all organs and tissues is critical to their physiological function and overall health. Capillaries facilitate blood supply, oxygenation and removal of metabolites from all organs. Anatomically, they are generally composed of endothelial cells, smooth muscle cells, and pericytes. The latter “monitor” and maintain the function and anatomical integrity of all capillaries.

When pathological processes such as diabetes mellitus, inflammatory, auto-immune, degenerative, age-related, trauma and other injuries affect the function of the capillary complex, blood supply, tissue oxygenation and removal of metabolites from affected organs is impaired, translating into permanent loss of function and systemic morbidity and mortality.

Mesenchymal Stem or Stromal Cells (MSC) from bone marrow, fat, umbilical cord and other sources are non-embryonic, “adult” stem cells that possess potent anti-inflammatory, anti-apoptotic, immune modulating, angiogenic and vasculo-protective, anti-fibrotic and anti-thrombotic paracrine activities that have been used for the promising treatment of various acute and chronic diseases and organ injuries. MSCs in culture release beneficial cytokines and growth factors that mediate their pleiotropic effects. In addition, MSCs, like essentially all cells, release Exosomes (30-100 nm in diameter) into their microenvironment that are taken up my adjacent cells, which, in turn, results in paracrine signaling in the target cells. Signaling occurs by the lateral transfer of mRNAs, miRNAs proteins, and lipids, which together are known to exert beneficial effects that parallel those of intact MSCs. Due to their small nanometer size and the ability to readily collect therapeutic quantities, Exosomes are particularly suited for the treatment of the diseased microvasculature into which larger MSCs (˜100 μm in diameter) can not and should not be delivered. It is known that the forced delivery of large cells such as MSCs into capillaries in which blood flow is significantly compromised because the lumens are pathologically reduced results in microinfarcts and accelerated tissue injury. The here disclosed technology harnesses the organ and vasculoprotective functions of MSCs, where appropriate, and of their exosomes where needed in order to repair and protect the affected capillary system in the diseases and disorders listed above.

### BRIEF SUMMARY

Described herein are a) mesenchymal and/or adipose stem cells; and b) mesenchymal and/or adipose stem cell-derived exosomes.

Also described are methods of treating a subject with microvascular disease, the methods comprising: administering to the subject (e.g. by injection) the mesenchymal and/or adipose stem cells or exosomes individually or in combination.

In this disclosure, the term Mesenchymal Stem Cell means and includes mesenchymal and/or adipose stem cells; and b) mesenchymal and/or adipose stem cell-derived exosomes. “Treating” or “treatment” does not require a complete cure. It means that the symptoms of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease.

As used herein “thereapeutically effective amount” is an amount sufficient to act as a treatment as defined above. This may be determined by, e.g., standard techniques used to monitor and/or diagnose a particular disease state.

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.

As used herein, a “subject” is typically a mammal.

### BRIEF DESCRIPTION OF THE DRAWINGS

While this disclosure concludes with claims particularly pointing out and distinctly claiming specific embodiments, various features and advantages of embodiments within the scope of this disclosure may be more readily ascertained from the following description when read in conjunction with the accompanying drawings, in which:

FIG. 1. The top panel illustrates the fact that patent capillaries can accommodate relatively large Mesenchymal Stem Cells (˜100 μm diameter), while capillaries with microvascular disease do not (bottom panel) presumably due to the pathologically reduced diameter of the affected capillary beds. In these Mesenchymal Stem Cell-derived very small Exosomes (˜40-100 nm diameter) that pleiotropically modulate the underlying pathomechanisms can be introduced to stabilize and repair the affected microvasculature. This therapy, in turn, translates into improved organ function in subjects with diabetes mellitus-associated end organ damage, vasculitis, toxic and ischemic organ injuries, sepsis, aging, atherosclerosis, retinopathy and/or others.

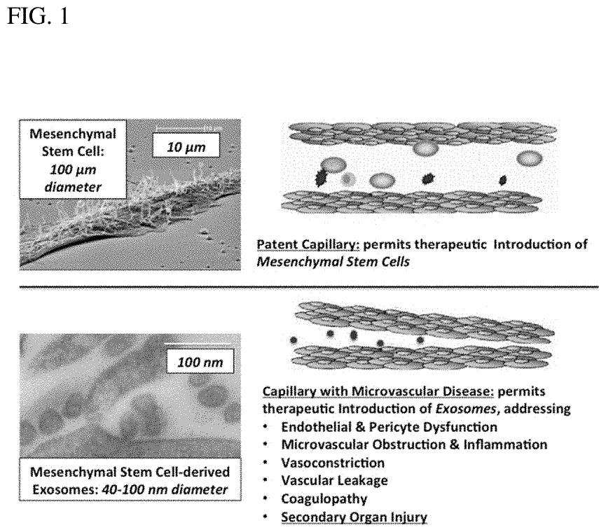

### EXAMPLES

The following examples are provided for illustration purposes only and are not to be construed as limiting the disclosure to the embodiments specifically disclosed therein.

Since both type 1 and type 2 diabetes mellitus progressively result in wide-spread end organ damage, affecting the retina, coronaries, the nervous system, kidneys and other organs though microvascular disease that injures the capillary beds in these organs through endothelial and pericytes dysfunction, microvascular obstruction and inflammation, vasoconstriction, vascular leakage, coagulopathy, and fibrosis. This microvascular damage also affects the capillaries of the islets of Langerhans in the pancreas. This pathomechanism could accelerate the destruction of insulin producing cells and thereby hasten the complete loss of intrinsic insulin production, i.e., greatly aggravate the diabetic state in a patient. Accordingly, we tested whether the parenteral administration of mesenchymal and/or adipose-derived exosomes to db/db mice with advanced diabetic disease would improve glycemic control. This was observed, together with improvement in diabetic kidney disease.

In analogy to the microvascular disease of diabetes, which shares most pathologic features of various vasculitides, auto-immune diseases, sepsis, acute and chronic kidney diseases, cardiovascular and neurological diseases, solid organ transplantation, vascular rejection, wound healing, atherosclerosis, aging, and various degenerative retinal diseases, we expect that the treatment with Mesenchymal and/or Adipose Stem Cells alone or their exosomes alone or in combination with their parent stem cells will favorably affect outcomes in this large group of microvascular diseases.

A type 2 diabetic male subject presents with chronic kidney disease. MSCs are isolated from the subject and expanded in culture. The expanded MSCs are administered to the subject in an amount sufficient to improve the symptoms from which the subject is suffering that are related to microvasculature issues.

### REFERENCES WITH PROTOCOLS FOR ISOLATION AND EXPANSION OF MSC/ASC (INCORPORATED BY REFERENCE HEREIN)

1. Toegel F, Hu Z, Weiss K, Isaac J, Lange C, Westenfelder C: Administered mesenchymal stem cells protect against ischemic acute renal failure through differentiation-independent mechanisms. Am J Physiol Renal Physiol. 2005 July; 289(1):F31-42. Epub 2005 Feb. 15
2. Togel F, Weiss K, Yang Y, Hu Z, Zhang P, Westenfelder C: Vasculotropic, paracrine actions of infused mesenchymal stem cells are important to the recovery from acute kidney injury. Am J Physiol Renal Physiol. 2007 May; 292(5):F1626-35. Epub 2007 Jan. 9.

### REFERENCES WITH PROTOCOLS FOR ISOLATION OF MSC/ASC-DERIVED EXOSOMES (INCORPORATED BY REFERENCE HEREIN)

3. Lötvall J, Hill A F, Hochberg F et al.: Minimal experimental requirements for definition of extracellular vesicles and their functions: a position statement from the International Society for Extracellular Vesicles. Journal of Extracellular Vesicles 2014, 3: 26913—http://dx.doi.org/10.3402/jev.v3.26913
4. Bang C, Thum T: Exosomes: New players in cell=cell communication. Int J Biochem Cell Biol 2012; 44:2060-2064.
5. Kholina S, Ranghino A, Garnieri P et al.: Extracellular vesicles as new players in angiogenesis. Vasc Pharmacol 2016; S1537-1891:30105-3-101.

1. A composition comprising:
mesenchymal and/or adipose stem cells; and/or
exosomes isolated from mesenchymal and/or adipose stem cells.

2. (canceled)

3. A method of treating a subject suffering from microvascular disease, the method comprising:
administering to the subject a therapeutically effective amount of mesenchymal and/or adipose stem cells.

4. The method according to claim 3, further comprising:
treating the subject with exosomes isolated from mesenchymal and/or adipose stem cells.

5. The method according to claim 4, wherein the subject is treated concomitantly with the mesenchymal and/or adipose stem cells and the exosomes isolated from mesenchymal and/or adipose stem cells.

6. The method according to claim 4, wherein the subject is treated sequentially with the mesenchymal and/or adipose stem cells and the exosomes isolated from mesenchymal and/or adipose stem cells.

7. The method according to claim 3, wherein the mesenchymal and/or adipose stem cells are administered parenterally, systemically, or upstream in the vascular system from damaged microvasculature.

8. The method according to claim 3, wherein the mesenchymal and/or adipose stem cells are allogeneic to the subject.

9. The method according to claim 3, wherein the microvascular disease is diabetes related microvascular disease.

10. The method according to claim 3, wherein the microvascular disease is associated with vasculitides, auto-immune disease, sepsis, acute or chronic kidney diseases, cardiovascular disease, neurological disease, solid organ transplantation, vascular rejection, wound healing, atherosclerosis, aging, and/or degenerative retinal disease.

11. A method of treating a subject suffering from microvascular disease, the method comprising:
administering to the subject a therapeutically effective amount of exosomes isolated from mesenchymal and/or adipose stem cells.

12. The method according to claim 11, wherein the exosomes are administered parenterally, systemically, or upstream in the vascular system from damaged microvasculature.

13. The composition of claim 1, wherein the composition comprises mesenchymal stem cells and exosomes isolated from adipose stem cells

14. The composition of claim 1, wherein the composition comprises adipose stem cells and exosomes isolated from mesenchymal stem cells.

15. The method according to claim 4, wherein the method comprises administering to the subject mesenchymal stem cells and treating the subject with exosomes isolated from adipose stem cells

16. The method according to claim 4, wherein the method comprises administering to the subject adipose stem cells and treating the subject with exosomes isolated from mesenchymal stem cells.
